(19) United States
(12) Patent Application Publication
(10) Pub. No.: US 2025/0104321 A1
(43) Pub. Date: Mar. 27, 2025

(54) THREE-DIMENSIONAL IMAGE GENERATION METHOD FOR GENERATING AN IMAGE ACCORDING TO TWO IMAGES OF DIFFERENT TIMES, AND GENERATING A THREE-DIMENSIONAL IMAGE ACCORDINGLY

(71) Applicant: Qisda Corporation, Taoyuan City (TW)

(72) Inventors: Min-Hsiung Huang, Taoyuan City (TW); Chuang-Wei Wu, Taoyuan City (TW); Tsung-Hsun Wu, Taoyuan City (TW)

(73) Assignee: Qisda Corporation, Taoyuan city (TW)

(21) Appl. No.: 18/822,496

(22) Filed: Sep. 3, 2024

(30) Foreign Application Priority Data
Sep. 22, 2023 (CN) 202311235724.X

(51) Int. Cl. G06T 15/00; G06V 10/141; G06V 10/74

(52) U.S. Cl. CPC G06T 15/00; G06V 10/141; G06V 10/761

(57) ABSTRACT

A three-dimensional image generation method includes projecting a first light pattern to an object to generate a first image at a first time, capturing the first image, projecting a second light pattern to the object to generate a second image at a second time, capturing the second image, generating a third image corresponding a third time according to the first image and the second image, and generating a three-dimensional image of the object according to the first image, the second image and the third image. The first time precedes the second time, and the second time precedes the third time.

### BACKGROUND OF THE INVENTION

### 1. Field of the Invention

The disclosure is related to a three-dimensional image generation method, and more particularly, a three-dimensional image generation method used for generating an image according to two images of different times, and generating a three-dimensional image accordingly.

### 2. Description of the Prior Art

As technology advances, an increasing number of professionals are leveraging optical assistive devices to enhance operational convenience and precision. One such example can be found in the field of dentistry, where intraoral scanners are currently employed to aid dentists in oral examinations. These scanners are capable of capturing images within the oral cavity and transforming them into digital data, thereby facilitating dental professionals, such as dentists and dental technicians, in their diagnostic procedures and denture fabrication processes.

When utilizing an intraoral scanner to acquire dental images, the user is required to continuously maneuver the scanner due to the confined space within the oral cavity. This allows for the capture of multiple images, which are subsequently stitched together to generate a three-dimensional image.

However, it has been observed in practical applications that the three-dimensional images produced by intraoral scanners often exhibit inaccurate deformations, leading to subpar image quality. Upon analysis, it has been determined that the degradation in the quality of three-dimensional images is frequently attributable to factors such as the scanner being moved too swiftly or the user's hand exhibiting tremors. The amalgamation of multiple captured images often results in a decline in the quality of the resultant three-dimensional image. Consequently, there is a pressing need for suitable solutions within this field to enhance the quality of the generated three-dimensional images.

### SUMMARY OF THE INVENTION

An embodiment provides a three-dimensional image generation method, including projecting a first light pattern to an object to generate a first image at a first time, capturing the first image, projecting a second light pattern to the object to generate a second image at a second time, capturing the second image, generating a third image corresponding to a third time according to the first image and the second image, and generating a three-dimensional image of the object according to the first image, the second image and the third image. The first time precedes the second time, and the second time precedes the third time.

Another embodiment provides a three-dimensional image generation method, including projecting a first light pattern to an object to generate a first image at a first time, capturing the first image, projecting a second light pattern to the object to generate a third image at a third time, capturing the third image, generating a second image corresponding to a second time according to the first image and the third image, and generating a three-dimensional image of the object according to the first image, the second image and the third image. The first time precedes the second time, and the second time precedes the third time.

Another embodiment provides a three-dimensional image generation method, including projecting a first light pattern to an object to generate a second image at a second time, capturing the second image, projecting a second light pattern to the object to generate a third image at a third time, capturing the third image, generating a first image corresponding to a first time according to the second image and the third image, and generating a three-dimensional image of the object according to the first image, the second image and the third image. The first time precedes the second time, and the second time precedes the third time.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 illustrates a three-dimensional image generation system according to an embodiment.

FIG. 2 illustrates a plurality of images generated by scanning the object according to an embodiment.

FIG. 3 illustrates a flowchart of a three-dimensional image generation method according to an embodiment.

FIG. 4 and FIG. 5 are diagrams of the operations using the three-dimensional image generation method of FIG. 3.

FIG. 6 illustrates a flowchart of a three-dimensional image generation method according to another embodiment.

FIG. 7 and FIG. 8 are diagrams of the operations using the three-dimensional image generation method of FIG. 6.

FIG. 9 illustrates a flowchart of a three-dimensional image generation method according to another embodiment.

FIG. 10 and FIG. 11 are diagrams of the operations using the three-dimensional image generation method of FIG. 9.

### DETAILED DESCRIPTION

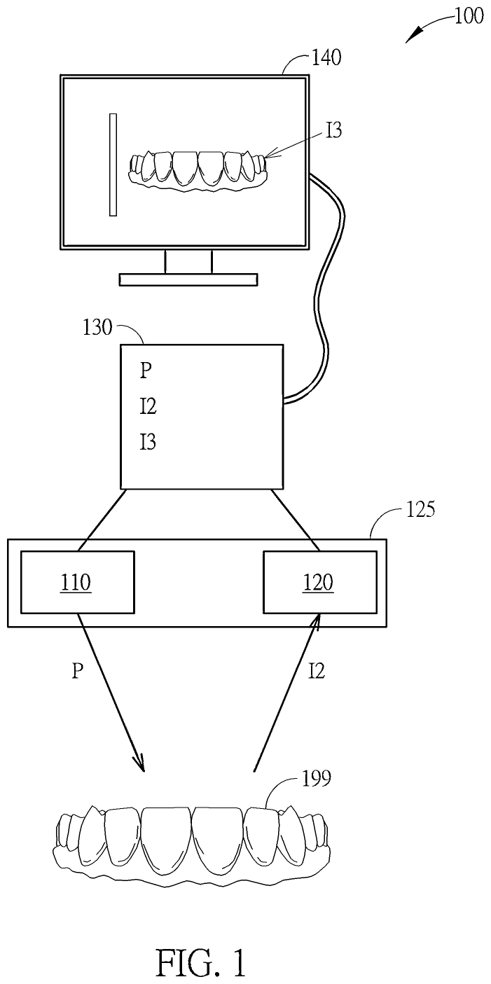

In the text, an intraoral scanner in the field of dentistry can be taken as an example for illustration. However, solutions provided by embodiments can also be applied to other fields and applications. FIG. 1 illustrates a three-dimensional image generation system 100 according to an embodiment. The 3D image generation system 100 can include a projector 110, a camera 120, a processor 130, and a display 140.

The projector 110 can be used to project a plurality of light patterns Ponto an object 199 (for example, teeth in an oral cavity) to generate a plurality of two-dimensional images I2. The camera 120 can be used to capture the two-dimensional images I2. The projector 110 and the camera 120 can be installed in a movable device 125, such as a handheld part of an intraoral scanner. The plurality of two-dimensional images I2 can be stitched together to generate a three-dimensional image I3.

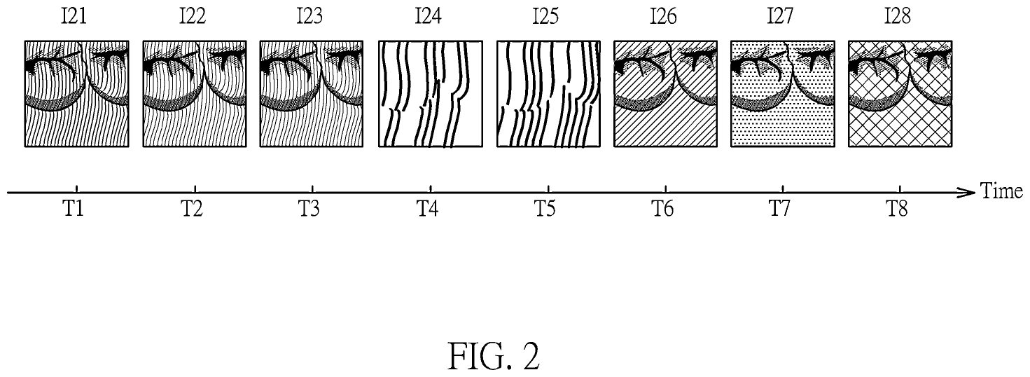

FIG. 2 illustrates a plurality of images generated by scanning the object 199 in an embodiment. In FIG. 2, the scanned object 199 can be teeth in an oral cavity. In FIG. 2, according to the order of time, eight two-dimensional images I21 to I28 can be captured sequentially. The images I21 to I28 can correspond to times T1 to T8 respectively, where time T1 can precede time T2, time T2 can precede time T3, time T3 can precede time T4, and so on. Time T7 can precede time T8.

For example, the images I21 to I25 can be obtained by projecting light patterns (such as stripes) to the object 199 and receiving the reflected light patterns. The images I26 to I28 can be obtained by projecting light of different colors (e.g. red, green, and blue, often abbreviated as R, G, and B) to the object 199 and receiving the reflected light.

In the example of FIG. 2, the images I21 to I25 can be used to generate the three-dimensional shape of the object 199. The images I21 to I23 can be generated by projecting scan lines to the object 199 and capturing the reflected lines. The images I24 and I25 can be generated by projecting mark lines to the object 199 and capturing the reflected lines. The mark lines can be used to confirm the positions of the scan lines in the image. The images I26 to I28 can be used to generate the colors and texture of the object 199 for viewing.

In FIG. 2, eight images (e.g. I21 to I28) can be captured and used to generate one three-dimensional image. However, the number of images in FIG. 2 is only an example. The quantity of two-dimensional images captured can be tailored to meet specific requirements.

In the event that the movable device 125 of FIG. 1 is operated at an excessively rapid pace, it may result in a series of two-dimensional images, produced by scanning the object 199, exhibiting substantial disparities. This could reduce the quality of the three-dimensional image I3, which is constructed through a stitching process. To illustrate, if the handheld component of the intraoral scanner is moved too swiftly, the content of the images I21, I22 and I23 (refer to FIG. 2) may exhibit significant variations. This could lead to complications such as distortion or damage in the three-dimensional image (for instance, the three-dimensional image I3 in FIG. 1) that is generated following the point cloud stitching process. To mitigate this issue, an additional two-dimensional image can be produced according to two previously captured two-dimensional images. These three two-dimensional images can then be utilized in the stitching process to construct a three-dimensional image. This approach effectively reduces the likelihood of producing low-quality three-dimensional images due to large disparities in the two-dimensional images. Further details related to this process are elaborated in the subsequent sections.

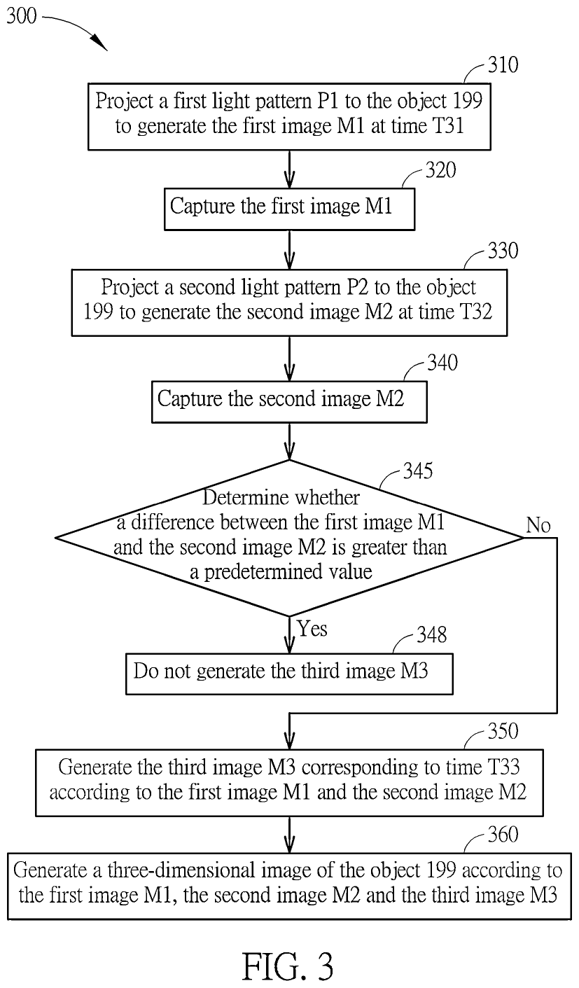

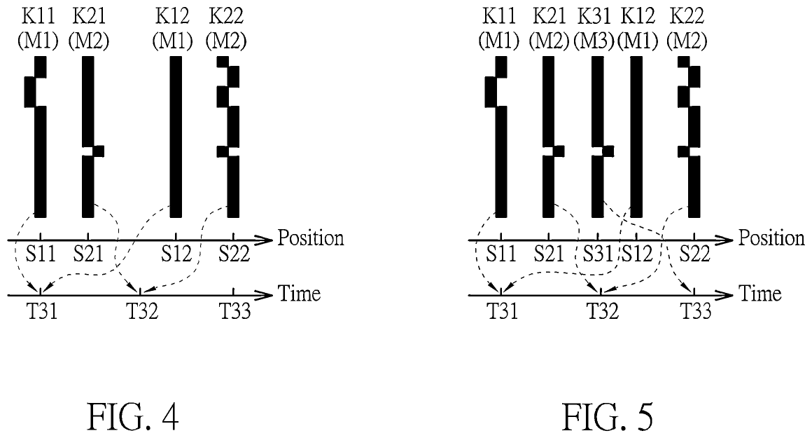

FIG. 3 illustrates a flowchart of a three-dimensional image generation method 300 in an embodiment. FIG. 4 and FIG. 5 are diagrams of the operations using the three-dimensional image generation method 300 of FIG. 3. FIG. 4 shows positions of stripes of a first image M1 and a second image M2. FIG. 5 further shows a position of a stripe of a third image M3. FIG. 4 and FIG. 5 serve merely as illustrations of the operational principles of the method in FIG. 3, and embodiments are not limited thereto. The three-dimensional image generation method 300 can include the following steps.
Step 310: project a first light pattern (expressed as P1 in the text) to the object 199 to generate the first image M1 at time T31;
Step 320: capture the first image M1;
Step 330: project a second light pattern (expressed as P2 in the text) to the object 199 to generate the second image M2 at time T32;
Step 340: capture the second image M2;
Step 345: determine whether a difference between the first image M1 and the second image M2 is greater than a predetermined value; if so, enter Step 348; otherwise, enter Step 350;
Step 348: do not generate the third image M3.
Step 350: generate the third image M3 corresponding to time T33 according to the first image M1 and the second image M2; and
Step 360: generate a three-dimensional image of the object 199 according to the first image M1, the second image M2 and the third image M3.

In FIG. 3, time T31 can precede time T32, and time T32 can precede time T33. The first light pattern P1 can be the same as the second light pattern P2. Step 345 and Step 348 can be optionally executed or omitted.

In Step 350, a coordinate position of a stripe of the third image M3 can be determined according to a coordinate position of a stripe of the first image M1 and a coordinate position of a stripe of the second image M2.

In FIG. 4, a stripe K11 can be a first stripe of the first image M1 captured at time T31, and its coordinate position can be S11. A stripe K12 can be a second stripe of the first image M1 captured at time T31, and its coordinate position can be S12. A stripe K21 can be a first stripe of the second image M2 captured at time T32, and its coordinate position can be S21. A stripe K22 can be a second stripe of the second image M2 captured at time T32, and its coordinate position can be S22.

As shown in FIG. 5, Step 350 can be performed to generate the position of a stripe K31 of the third image M3 according to the position of the stripe in the first image M1 and the position of the stripe in the second image M2. The coordinate position of the stripe K31 of the third image M3 can be S31 and corresponding to time T33.

For instance, at Step 350, the coordinate position S31 can be generated according to the coordinate positions S12 and S21. This relationship can be represented as S31=F(S12, S21), where F( ) can denote a function. For instance, the coordinate position S31 can be a half-sum of the coordinate positions S12 and S21, and it can be represented as S31=(S12+S21)/2.

In another embodiment, the coordinate position S31 can be generated according to the coordinate positions S11 and S21. This relationship can be represented as S31=G(S11, S21), where G( ) can denote a function. For instance, the coordinate position S31 can be a sum of the coordinate position S21 and a difference between the coordinate positions S21 and S11, and it can be represented as S31=S21+(S21−S11).

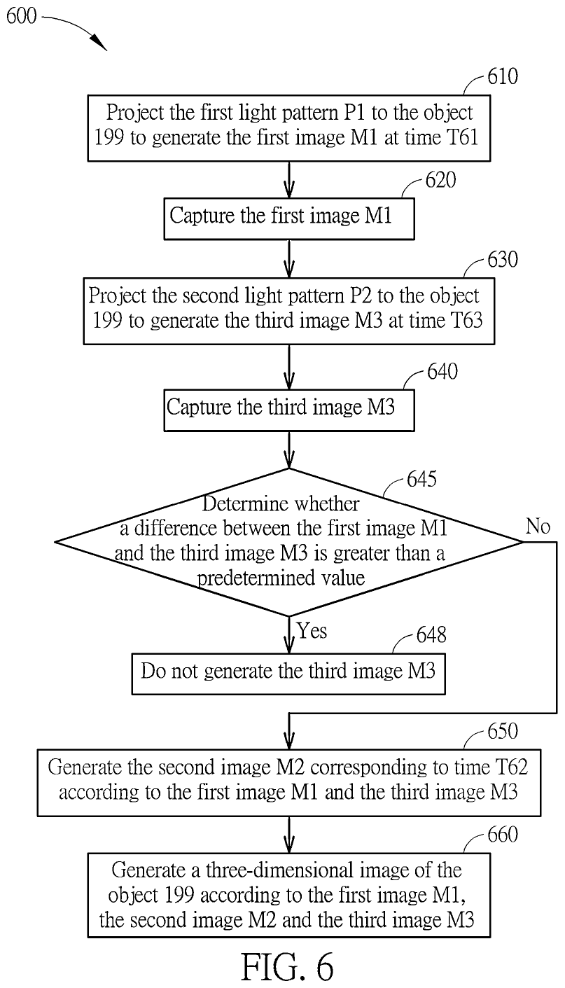

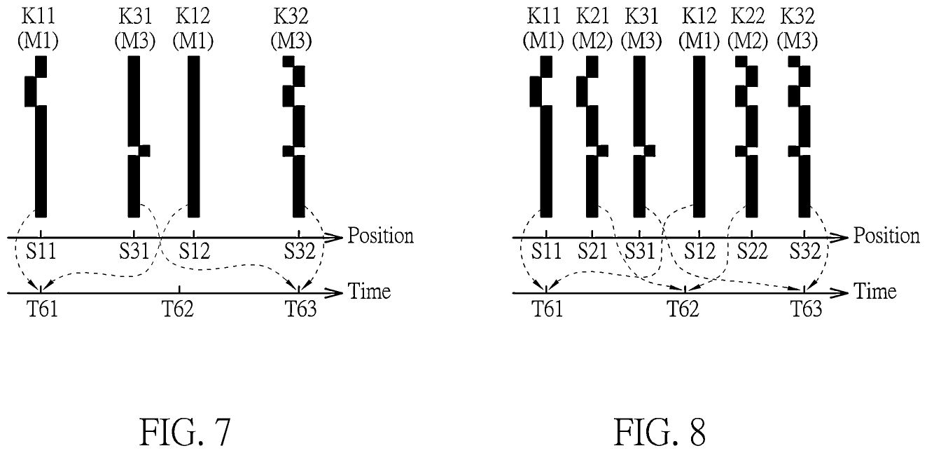

FIG. 6 illustrates a flowchart of a three-dimensional image generation method 600 according to an embodiment. FIG. 7 and FIG. 8 are diagrams of the operations using the three-dimensional image generation method 600 of FIG. 6. FIG. 7 shows positions of stripes of the first image M1 and the third image M3. FIG. 8 further shows positions of stripes of the second image M2. FIG. 7 and FIG. 8 serve merely as illustrations of the operational principles of the method in FIG. 6, and embodiments are not limited thereto. The three-dimensional image generation method 600 can include the following steps.
Step 610: project the first light pattern P1 to the object 199 to generate the first image M1 at time T61;
Step 620: capture the first image M1;
Step 630: project the second light pattern P2 to the object 199 to generate the third image M3 at time T63;
Step 640: capture the third image M3;
Step 645: determine whether a difference between the first image M1 and the third image M3 is greater than a predetermined value; if so, enter Step 648; otherwise, enter Step 650;
Step 648: do not generate the third image M3.
Step 650: generate the second image M2 corresponding to time T62 according to the first image M1 and the third image M3; and
Step 660: generate a three-dimensional image of the object 199 according to the first image M1, the second image M2 and the third image M3.

In FIG. 3, time T61 can precede time T62, and time T62 can precede time T63. The first light pattern P1 can be the same as the second light pattern P2. Step 645 and Step 648 can be optionally executed or omitted.

In Step 650, a coordinate position of a stripe of the second image M2 can be determined according to a coordinate position of a stripe of the first image M1 and a coordinate position of a stripe of the third image M3.

In FIG. 7, the stripe K11 can be the first stripe of the first image M1 captured at time T61, and its coordinate position can be S11. The stripe K12 can be the second stripe of the first image M1 captured at time T61, and its coordinate position can be S12. The stripe K31 can be the first stripe of the third image M3 captured at time T63, and its coordinate position can be S31. A stripe K32 can be a second stripe of the third image M3 captured at time T63, and its coordinate position can be S32.

As shown in FIG. 8, in Step 650, the position(s) of the first stripe K21 and/or the second stripe K22 of the second image M2 can be generated according to the positions of the stripes of the first image M1 and the third image M3. The coordinate position of the first stripe K21 of the second image M2 can be S21 and corresponding to time T62. The coordinate position of the second stripe K22 of the second image M2 can be S22 and also corresponding to time T62.

For instance, in Step 650, the coordinate position S21 can be generated according to the coordinate positions S11 and S31. This relationship can be represented as S21=H(S11, S31), where H( ) can denote a function. For instance, the coordinate position S21 can be a half-sum of the coordinate positions S11 and S31, and it can be represented as S21=(S11+S31)/2.

In another embodiment, in Step 650, the coordinate position S22 can be generated according to the coordinate positions S12 and S31. This relationship can be represented as S22=I(S12, S31), where I( ) can denote a function. For instance, the coordinate position S22 can be a sum of the coordinate position S12 and a difference between the coordinate positions S12 and S31, and it can be represented as S22=S12+(S12−S31).

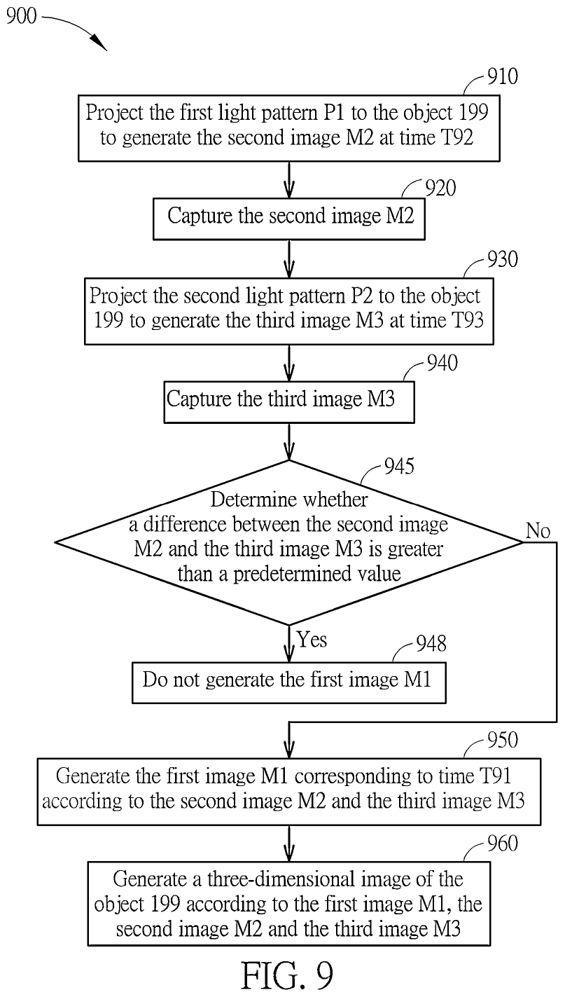

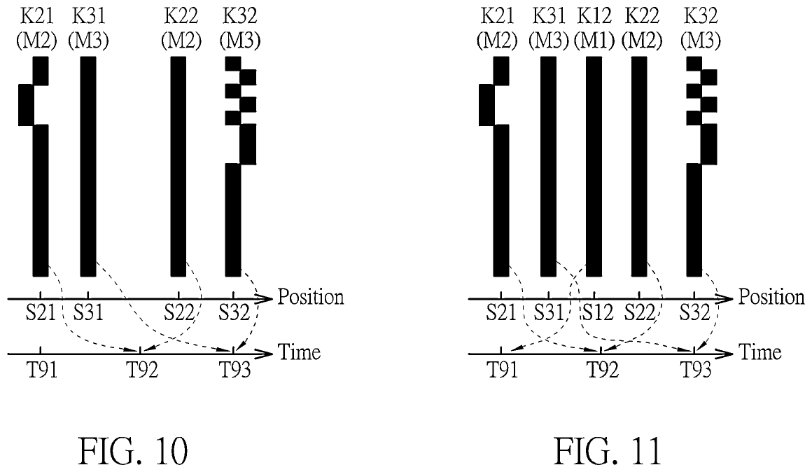

FIG. 9 illustrates a flowchart of a three-dimensional image generation method 900 according to an embodiment. FIG. 10 and FIG. 11 are diagrams of the operations using the three-dimensional image generation method 900 of FIG. 9. FIG. 10 shows positions of stripes of the second image M2 and the third image M3. FIG. 11 further shows a position of a stripe of the first image M1. FIG. 10 and FIG. 11 serve merely as illustrations of the operational principles of the method in FIG. 9, and embodiments are not limited thereto. The three-dimensional image generation method 900 can include the following steps.
Step 910: project the first light pattern P1 to the object 199 to generate the second image M2 at time T92;
Step 920: capture the second image M2;
Step 930: project the second light pattern P2 to the object 199 to generate the third image M3 at time T93;
Step 940: capture the third image M3;
Step 945: determine whether a difference between the second image M2 and the third image M3 is greater than a predetermined value; if so, enter Step 948; otherwise, enter Step 950;
Step 948: do not generate the first image M1.
Step 950: generate the first image M1 corresponding to time T91 according to the second image M2 and the third image M3; and
Step 960: generate a three-dimensional image of the object 199 according to the first image M1, the second image M2 and the third image M3.

In FIG. 9, time T91 can precede time T92, and time T92 can precede time T93. The first light pattern P1 can be the same as the second light pattern P2. Step 945 and Step 948 can be optionally executed or omitted.

In Step 950, a coordinate position of a stripe of the first image M1 can be determined according to a coordinate position of a stripe of the second image M2 and a coordinate position of a stripe of the third image M3.

In FIG. 10, the stripe K21 can be the first stripe of the second image M2 captured at time T92, and its coordinate position can be S21. The stripe K22 can be the second stripe of the second image M2 captured at time T92, and its coordinate position can be S22. The stripe K31 can be the first stripe of the third image M3 captured at time T93, and its coordinate position can be S31. The stripe K32 can be the second stripe of the third image M3 captured at time T93, and its coordinate position can be S32.

As shown in FIG. 11, in Step 950, the position of the stripe K12 of the first image M1 can be generated according to the positions of the stripes of the second image M2 and the third image M3. The coordinate position of the stripe K12 of the first image M1 can be S12 and corresponding to time T91.

For instance, in Step 950, the coordinate position S12 can be generated according to the coordinate positions S22 and S31. This relationship can be represented as S12=J(S22, S31), where J( ) can denote a function. For instance, the coordinate position S12 can be a half-sum of the coordinate positions S31 and S22, and it can be represented as S12=(S31+S22)/2.

In another embodiment, in Step 950, the coordinate position S12 can be generated according to the coordinate positions S21 and S31. This relationship can be represented as S12=K(S21, S31), where K( ) can denote a function. For instance, the coordinate position S12 can be a sum of the coordinate position S31 and a difference between the coordinate positions S31 and S21, and it can be represented as S12=S31+(S31−S21).

In accordance with the above, two images corresponding to two distinct time points can be captured initially. Using the coordinate positions of the stripes in these two images, a stripe corresponding to a different image at a different time can be produced. Subsequently, a three-dimensional image of the object can be generated using the stripes from these three images. FIG. 3, FIG. 4 and FIG. 5, FIG. 6, FIG. 7 and FIG. 8, and FIG. 9, FIG. 10 and FIG. 11 merely serve as examples for describing the operational principles of embodiments. During actual operation, each of the two known stripes (such as K21 and K12 in FIG. 5) can possess multiple points, hence each stripe can be corresponding to several coordinate positions. As a result, the stripe that is generated accordingly (like K31 in FIG. 5) can also encompass multiple coordinate positions. These coordinate positions can be generated according to the coordinate positions of the two known stripes.

In conclusion, the three-dimensional image generation system 100, as well as the three-dimensional image generation methods 300, 600 and 900, enable the generation of an additional image using two pre-existing images, followed by the creation of a three-dimensional image utilizing these three images. This approach effectively mitigates the issue of excessive discrepancies in two-dimensional images, which may arise due to rapid movement of the detection apparatus (such as the mobile device 125 depicted in FIG. 1). Consequently, this results in a significant enhancement in the quality of the three-dimensional image produced by stitching two-dimensional images.

Those skilled in the art will readily observe that numerous modifications and alterations of the device and method may be made while retaining the teachings of the invention. Accordingly, the above disclosure should be construed as limited only by the metes and bounds of the appended

What is claimed is:

1. A three-dimensional image generation method comprising:
projecting a first light pattern to an object to generate a first image at a first time;
capturing the first image;
projecting a second light pattern to the object to generate a second image at a second time;
capturing the second image;
generating a third image corresponding to a third time according to the first image and the second image; and
generating a three-dimensional image of the object according to the first image, the second image and the third image;
wherein the first time precedes the second time, and the second time precedes the third time.

2. The method of claim 1, wherein the first light pattern is the same as the second light pattern.

3. The method of claim 1, wherein generating the third image corresponding to the third time according to the first image and the second image comprises:
generating a coordinate position of a stripe of the third image according to a coordinate position of a stripe of the first image and a coordinate position of a stripe of the second image.

4. The method of claim 1, wherein the coordinate position of the stripe of the first image is S12, the coordinate position of the stripe of the second image is S21, the coordinate position of the stripe of the third image is S31, and S31=(S12+S21)/2.

5. The method of claim 1, wherein the coordinate position of the stripe of the first image is S11, the coordinate position of the stripe of the second image is S21, the coordinate position of the stripe of the third image is S31, and S31=(S21−S11)+S21.

6. The method of claim 1, further comprising:
determining whether a difference between the first image and the second image is greater than a predetermined value;
wherein the third image is generated if the difference between the first image and the second image is less than the predetermined value.

7. A three-dimensional image generation method comprising:
projecting a first light pattern to an object to generate a first image at a first time;
capturing the first image;
projecting a second light pattern to the object to generate a third image at a third time;
capturing the third image;
generating a second image corresponding to a second time according to the first image and the third image; and
generating a three-dimensional image of the object according to the first image, the second image and the third image;
wherein the first time precedes the second time, and the second time precedes the third time.

8. The method of claim 7, wherein the first light pattern is the same as the second light pattern.

9. The method of claim 7, wherein generating the second image corresponding to the second time according to the first image and the third image comprises:
generating a coordinate position of a stripe of the second image according to a coordinate position of a stripe of the first image and a coordinate position of a stripe of the third image.

10. The method of claim 9, wherein the coordinate position of the stripe of the first image is S11, the coordinate position of the stripe of the third image is S31, the coordinate position of the stripe of the second image is S21, and S21=(S11+S31)/2.

11. The method of claim 9, wherein the coordinate position of the stripe of the first image is S12, the coordinate position of the stripe of the third image is S31, the coordinate position of the stripe of the second image is S22, and S22=S12+(S12−S31).

12. The method of claim 7, further comprising:
determining whether a difference between the first image and the third image is greater than a predetermined value;
wherein the second image is generated if the difference between the first image and the third image is less than the predetermined value.

13. A three-dimensional image generation method comprising:
projecting a first light pattern to an object to generate a second image at a second time;
capturing the second image;
projecting a second light pattern to the object to generate a third image at a third time;
capturing the third image;
generating a first image corresponding to a first time according to the second image and the third image; and
generating a three-dimensional image of the object according to the first image, the second image and the third image;
wherein the first time precedes the second time, and the second time precedes the third time.

14. The method of claim 13, wherein the first light pattern is the same as the second light pattern.

15. The method of claim 13, wherein generating the first image corresponding to the first time according to the second image and the third image comprises:
generating a coordinate position of a stripe of the first image according to a coordinate position of a stripe of the second image and a coordinate position of a stripe of the third image.

16. The method of claim 15, wherein the coordinate position of the stripe of the second image is S22, the coordinate position of the stripe of the third image is S31, the coordinate position of the stripe of the first image is S12, and S12=(S31+S22)/2.

17. The method of claim 15, wherein the coordinate position of the stripe of the second image is S21, the coordinate position of the stripe of the third image is S31, the coordinate position of the stripe of the first image is S12, and S12=S31+(S31−S21).

18. The method of claim 13, further comprising:
determining whether a difference between the second image and the third image is greater than a predetermined value;
wherein the first image is generated if the difference between the second image and the third image is less than the predetermined value.
